(19) United States
(12) Patent Application Publication
(10) Pub. No.: US 2004/0161459 A1
(43) Pub. Date: Aug. 19, 2004

(54) Fast-dissolve tablet technology

(76) Inventors: Ngoc Do, Bel Air, MD (US); Cheng Der Tony Yu, Baltimore, MD (US); Larry Augsburger, Severna Park, MD (US)

Correspondence Address: UPM PHARMACEUTICALS INC., 6200 SEAFORTH STREET, BALTIMORE, MD, 21224, US

(21) Appl. No.: 10/633,445

(22) Filed: Aug. 1, 2003

Related U.S. Application Data
(60) Provisional application No. 60/437,507, filed on Dec. 31, 2002.

(51) Int. Cl. A61K 9/20; D04H 1/00

(52) U.S. Cl. 424/465; 264/109

(57) ABSTRACT

The current invention relies on a process already provided in great detail in U.S. Pat. No. 60/437,507 and an associated CIP filed on May 23, 2003 and hereby incorporated by reference. However, it was noted during additional studies that if the Cushioning Beads™ were milled to a particle size between about 10 mesh and about 50 mesh and to a most preferred size of 30-40 mesh, the Cushioning Beads™ did not loose their ability to protect coated particles during compression, as would be expected because of the fine milling. Moreover, it was also discovered that tablets compressed from the milled Cushioning Beads™ immediately disperse in the mouth. A final advantage of the current invention is the improved hardness and friability. The invention produces a tablet of 2Kp or 20N hardness. A vast improvement because it allows for use of conventional manufacturing and packaging equipment.

### PRIORITY

This application is a Continuation-In-Part of application Ser. No. 60/437,507 filed on Dec. 31, 2002. This application claims priority back to the application Ser. No. 60/437,507 and incorporates said application by reference.

### BACKGROUND

In recent years, there has been developing interest in tablets that disperse rapidly in the mouth without requiring any water intake other than the normal flow of saliva. Such tablets make it easier for many elderly and children who often have difficulty in chewing or swallowing large capsules or tablets to take their medication. Such fast-dispersion tablets are also more convenient for anyone who is active and may not have convenient access to water for swallowing conventional dosage forms. Finally, because fast-disperse tablets are never swallowed whole, they may be particularly convenient dosage forms for delivering larger drug doses for any patient. Thus even large doses of drug that would otherwise require excessively large single tablets or capsules or the administration multiple tablets or capsules at one time may be conveniently administered in a single rapid-disperse tablet that does not need to be swallowed whole.

The primary challenges in developing rapid-disperse tablets are: 1) achieving palatability and 2) achieving robustness defined as adequate hardness and resistance to chipping and abrasion upon packaging and handling without creating an unduly increasing the disintegration time.

When necessary, palatability of drugs can generally be enhanced through the application of existing technologies; for example, those that involve coating and/or microencapsulation or formulation means.

Achieving robustness with short disintegration times in compressed tablets is more difficult since such tablets must not be compressed too hard to avoid prolonging disintegration, yet, at the same time, adequate compression must be used to produce tablets of practical hardness that would permit the tablets to be handled, shipped, and carried by patients. Currently the disintegration time can be characterized by either in vivo or in vitro tests using a timer with a desirable time range of 0-60 seconds, a preferred range of 0-30 seconds, and a most preferred range of 0-20 seconds. This invention describes pharmaceutical compositions which are comprised of a highly compactable, rapidly dispersing tablet matrix and one or more pharmaceutically active compounds that may be directly compressed to form rapidly dispersing tablets with practical hardness and resistance to chipping and abrasion. Thereby, the current invention overcomes many of the current manufacturing and packaging issues associated with other fast-dissolve tablets noted is U.S. Pat. Nos. 6.221,392 and 5,223,264. In addition, the current invention does not rely on an effervescent material and therefore differs significantly from U.S. Pat. Nos. 5,223,264; 5,639,475 and 5,807,577. The technology is also not limited by particle size, as is the case with U.S. Pat. No. 4,533,543. If necessary, the pharmaceutically active compounds may be made suitably palatable by coating or microencapsulation or by use any other formulation technology.

### DETAILED DESCRIPTION OF THE INVENTION

The current invention relies on a process already provided in great detail in U.S. Pat. No. 60/437,507 and an associated CIP filed on May 23, 2003 and hereby incorporated by reference. However, it was noted during additional studies that if the Cushioning Beads™ were milled to a particle size between about 10 mesh and about 50 mesh and to a most preferred size of 30-40 mesh, the Cushioning Beads™ did not loose their ability to protect coated particles during compression, as would be expected because of the fine milling. Moreover, it was also discovered that tablets compressed from the milled Cushioning Beads™ immediately disperse in the mouth. A final advantage of the current invention is the improved hardness and friability. The invention produces a tablet of 2Kp or 20N hardness. A vast improvement because it allows for use of conventional manufacturing and packaging equipment. These observations led to a very unexpected advantage of the Cushioning Beads®0 technology, that of forming tablet compositions that rapidly disperse and dissolve in the mouth. This discovery provides a very important formulating and marketing advantage.

The manufacturing process, according to this invention, allows the active and the cushioning components to be made into a single component at the ratio of active to cushioning ranging from 0.1% to 97%. The resultant granules, which consist of the active beads embedded within a layer of porous cushioning material, provide protection for the active-loaded beads during compression, see FIG. 1.

The process can be summarized in five general phases: the manufacture and coating of active-loaded beads with a sustained release coat, an enteric coat; a colonic coat, or a taste-masking coat; the manufacture of the cushioning components the co-processing of active-loaded beads with cushioning components into a single component known as a Cushioning Bead™; the use of freeze-drying to produce the intended outcome, and the compression of the resultant Cushioning Beads™ into tablets (see FIG. 2).

An alternative method of manufacture is to not co-process the active-loaded beads with cushioning components rather to direct blend the milled freeze-dried Cushioning Beads™ with a taste-masked, sustained-release or enterically-coated active followed by compression into a tablet (see FIG. 3) the alternative production method flowchart.

Manufacture and Coating of Active-Loaded Beads with a Sustained Release Coat, an Enteric Coat, a Colonic Coat, or a Taste-Masking Coat

Biologically active ingredients are contained in the active-loaded beads. The configuration of the active-loaded beads can be either a matrix, in which the biologically active ingredients are distributed throughout the inactive pharmaceutical excipients, or a drug-layered bead, in which layers of the biologically active ingredients are deposited around an inert nonpareil seed. In addition, the active-loaded bead can contain more than one active pharmaceutical ingredient. For the former, an extrusion/spheronization process is employed. A moistened, well-mixed mass of active and inactive ingredients is extruded into strands and subsequently rounded into spheroids or pellets in a spheronizer and dried in an oven or a fluid-bed dryer. A typical formulation for extrusion-spheronization consists of microcrystalline cellulose in combination with lactose, starch and other appropriate pharmaceutical excipients. For the latter, the biologically active ingredients are dispersed in a binder solution that can be layered onto nonpareil seeds using a typical fluid-bed coater. The binder solution includes but is not limited to appropriate levels of such common polymeric binders as hydroxypropyl-methylcellulose and polyvinylpyrrolidone.

The active-loaded beads are further coated with functional polymers to achieve a sustained-release delivery, an enteric delivery, a colonic delivery, or taste masking. Different polymers are used, depending on the objective of the drug delivery. Two classes of polymers are commonly used for a sustained release coating are the cellulosic polymers and methacrylate ester copolymers. Examples of these polymers include but are not limited to Eudragit® NE, Eudragit® RS/RL, Aquacoat® and Surelease®. Other excipients such as plasticizers, secondary polymers, water-soluble and water-insoluble additives are often included in the formulation to facilitate manufacture and/or achieve a desired dissolution profile. Enteric polymers are employed to prevent the contact of the biologically active ingredients with gastric juice and to facilitate the release of the drug in the small intestine region of the GI tract. Examples of the enteric polymers are Eudragit® L and S, Aquateric® and Sureteric®. Certain grades of these polymers may be selected to target release in specific regions of the intestine or colon based on the pH at which they are soluble.

Manufacture of the Cushioning Component

The cushioning component consists of a highly-compactable filler, such as microcrystalline cellulose, in combination with a highly water-absorbing material, such as Ac-Di-Sol®. Disintegrants and superdisintegrants, such as starch, croscarmellose sodium, crospovidone, and sodium starch glycolate, or hydrophilic materials, such as hydroxypropyl cellulose, can be used as the highly water-absorbing material. The highly water-absorbable materials can range from 5 to 90% (w/w).

The cushioning components are dry-blended and then granulated in a planetary mixer via a typical low-shear wet granulation process with purified water as a granulating fluid. The end-point of granulation of the cushioning components is reached when visual inspection confirms that granules are produced.

Co-Processing of Cushioning Components and Active-Loaded Beads

Sustained-release coated, colonic coated, enteric coated, or taste-masking coated active-loaded beads are generally added to the granulated cushioning components to produce the Cushioning Beads™. The active-loaded beads can alternatively be added to the cushioning dry-powder blend prior to the wet granulation step. The moistened granules of the well dispersed, active-loaded beads in the cushioning components are either passed through a screen of appropriate size or extruded and spheronized. The beads, pellets or granules thus obtained are then freeze-dried. It is understood in this disclosure that Cushioning Beads™ includes all beads, pellets or granules made in accordance with methods and art disclosed herein.

Freeze-Drying of the Cushioning Beads™

The co-processed Cushioning Beads™ are then placed into a freeze-dryer until a Loss on Drying (LOD) of less than 5% is achieved. Upon achievement of the LOD, the Cushioning Beads™ can be placed through a sieve to remove fines or move directly to the tableting process.

The freeze-drying creates the unexpected cushioning characteristic of the beads or pellets and produces a very porous layer which surrounds the active-loaded beads. Protected by the high porosity cushioning layer, the coatings of the active-loaded beads can withstand compression pressure during the tableting process as high as 1000 kg or more, depending on the nature of the coated beads and the proportion, particle size and composition of the Cushioning Beads™ in the tableting mixture. In addition to the cushioning characteristic, the freeze-drying creates a non-hygroscopic Cushioning Bead™ that does not require any special handling or packaging. Thereby overcoming many of the disadvantages associated with U.S. Pat. No. 6,221,392 and the increased costs associated with the use of the existing technology.

Milling of Cushioning Beads™

Upon freeze-drying the Cushioning Beads™ are then milled to 10-325 mesh (2000-45 micron), with a preferred size of 20-45 mesh and a most preferred size mesh of 30-40. This step increases the surface area of the Cushioning Beads™ and allows the final tablet to rapid disperse on the tongue without the addition of a liquid other than normal saliva.

Compression of the Freeze-Dried Cushioning Beads™ into Tablets

The compression of the final product, the Cushioning Bead™, follows a normal tablet compression operation. An additional advantage of the current invention is that no additional extra-granular ingredient, especially the binder, is required, because of the inter-locking mechanism created by the deformation of the cushioning layer during compression. The resultant tablets not only can maintain their mechanical strength but also can disintegrate rapidly upon contact with water in less than 10 seconds depending on the amount of active.

This invention also encompasses prodrug derivatives of the compounds contained herein. The term “prodrug” refers to a pharmacologically inactive derivative of a parent drug molecule that requires biotransformation, either spontaneous or enzymatic, within the organism to release the active drug. Prodrugs are variations or derivatives of the compounds of this invention which have groups cleavable under metabolic conditions. Prodrugs become the compounds of the invention which are pharmaceutically active in vivo, when they undergo solvolysis under physiological conditions or undergo enzymatic degradation. Prodrug compounds of this invention may be called single, double, triple etc., depending on the number of biotransformation steps required to release the active drug within the organism, and indicating the number of functionalities present in a precursor-type form. Prodrug forms often offer advantages of solubility, tissue compatibility, or delayed release in the mammalian organism (see, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, Calif., 1992). Prodrugs commonly known in the art include acid derivatives well known to practitioners of the art, such as, for example, esters prepared by reaction of the parent acids with a suitable alcohol, or amides prepared by reaction of the parent acid compound with an amine, or basic groups reacted to form an acylated base derivative. Moreover, the prodrug derivatives of this invention may be combined with other features herein taught to enhance bioavailability. The preparation of pharmaceutically acceptable isomers, solvates or hydrates would be apparent to one of ordinary skill in the art.

Glossary

Cushioning Beads™: As originally disclosed in U.S. Pat. No. 5,078,055, Cushioning Beads™ are spherical, semi-spherical agglomerates of suitable composition with a structure and deformation property such that when present in suitable proportion in admixture with membrane coated active-loaded beads and the admixture compressed to form a pharmaceutical tablet, the cushioning beads deform preferentially (that is, they deform at lower pressures) to substantially prevent rupture or cracking of the membrane of the active-loaded beads. Generally, cushioning beads do not contain a biologically active substance. Aulton et al. [Drug Development and Industrial Pharmacy, Vol. 20, pp. 3069-3104 (1994), at page 3094] refers to them as ‘placebo millispheres.’ Mount et al. [Drug Development and Industrial Pharmacy, Vol. 22,pp.609-612 (1996), at page 612] refers to them a ‘cushioning agents.’ The definition of cushioning beads is now expanded to include those active-loaded beads co-processed with cushioning components under current invention as demonstrated in FIG. 1.

Pharmaceutical composition: A pharmaceutical composition is a designed pharmaceutical formulation assembled (processed) in such a way as to meet certain functional criteria (e.g. appropriate drug release characteristics, stability, manufacturability, patient acceptability, content uniformity). Biologically active substances are seldom administered alone, but rather as part of a pharmaceutical composition or formulation in combination with one or more non-medical ingredients called excipients that serve varied and specialized functions, such as fillers, binders, lubricants, glidants, inert core beads, release rate-controlling components, stabilizers, flavors, colors, and others. The selection of excipients and their levels in the formulation, the method of assembly, and the appropriate adjustment of process variables together determine how closely the pharmaceutical composition meets its design criteria.

LOD: Loss on drying is defined as the percentage of water removed when a material is dried in an oven, or under infrared light, with or without the aid of a vacuum Cushioning Component: The cushioning component consists of a highly-compactable filler, such as microcrystalline cellulose, in combination with a highly water-absorbing material, such as Ac-Di-Sol®. Disintegrants and superdisintegrants, such as starch, croscarnellose sodium, crospovidone, and sodium starch glycolate, or hydrophilic materials, such as hydroxypropyl cellulose, can be used as the highly water-absorbing material

Patient: a mammal, preferably a human, in need of treatment of a condition, disorder or disease.

Treat and Treatment: Refer to both therapeutic treatments and prophylactic or preventative measures, wherein the object is to prevent or slow down (lessen) an undesired physiological condition, disorder or disease or obtain beneficial or desired clinical results. For purposes of this invention, beneficial or desired clinical results include but are not limited to, alleviation of symptoms; diminishment of extent of condition, disorder or disease; stabilized (i.e. not worsening) state of condition, disorder or disease; delay or slowing of condition, disorder or disease progression; amelioration of the condition, disorder or disease state; remission (whether partial or total), whether detectable or undetectable; or enhancement or improvement of condition, disorder or disease. Treatment includes eliciting a cellular response that is clinically significant, without excessive levels of side effects. Treatment also includes prolonging survival as compared to expected survival if not receiving treatment.

Mammal: Refers to any animal classified as a mammal, including humans, domestic and farm animals, and zoo, sports and pet companion animals such as household pet and other domesticated animals such as, but not limited to, cattle, sheep, ferrets, swine, horses, poultry, rabbits, goats, dogs, cats and the like. Preferred companion animals are dogs and cats. Preferably, the mammal is human.

Biological property: for the purposes herein means an in vivo effector or antigenic function or activity that is directly or indirectly performed by a compound of this invention that are often shown by in vitro assays. Effector functions include receptor or ligand binding, any enzyme activity or enzyme modulatory activity, any carrier binding activity, any hormonal activity, any activity in promoting or inhibiting adhesion of cells to an extracellular matrix or cell surface molecules, or any structural role. Antigenic functions include possession of an epitope or antigenic site that is capable of reacting with antibodies raised against it.

Active Pharmaceutical Ingredient: The biologically active ingredient in any pharmaceutical composition. The “API” is the ingredient that creates the desired biological property in the patient in need of treatment.

Pharmaceutically Acceptable Salts: includes salts of compounds derived from the combination of a compound and an organic or inorganic acid. These compounds are useful in both free base and salt form. In practice, the use of the salt form amounts to use of the base form; both acid and base addition salts are within the scope of the present invention.

Fast-Dissolve Tablet: A disintegration time on the tongue with the addition of no liquid of between 0-60 seconds with the most preferred disintegration time of 0-20 seconds.

Tablet Hardness: A measure of the physical strength of a tablet, hardness is the breaking force of a tablet measured in a consistent way using a suitable mechanized pharmaceutical hardness tester. Hardness testers suitable for testing and verifying the specifications in this disclosure areavailable from such laboratory supply houses such as VanKel Technology Group, 13000 Weston Parkway, Cary, N.C. 27513, or from Erweka Instruments, Inc., 56 Quirk Road, Milford, Conn. 06460

Friability: Friability is another measure of the physical strength of a compressed tablet based on its ability to resist fracture, chipping and abrasion in a specially designed device. Friability test equipment (also known as friabilators) suitable for testing and verifying the specifications described herein are available from such laboratory supply houses as VanKel Technology Group, 13000 Weston Parkway, Cary, N.C. 27513, or from Erweka Instruments, Inc., 56 Quirk Road, Milford, Conn. 06460

Active-Loaded Bead: The API coated with an appropriate coating to achieve a desired result such as enteric, sustained release or taste-masking.

Therapeutic Applications

The described drug delivery technology would be applicable for any active pharmaceutical ingredient of choice wherein the desired effect is a fast-dissolve on the tongue with the addition of no liquid other than saliva.

### FIGURES

FIG. 1: Cross section of Cushioning Beads™ containing the active loaded bead.

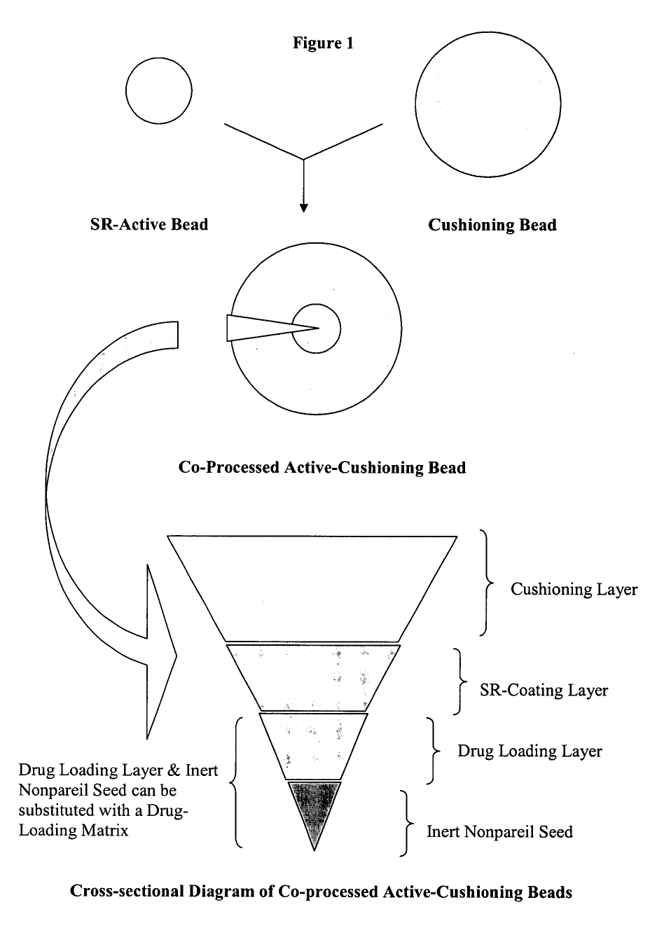

FIG. 2: Flow chart of the manufacturing process for co-processing.

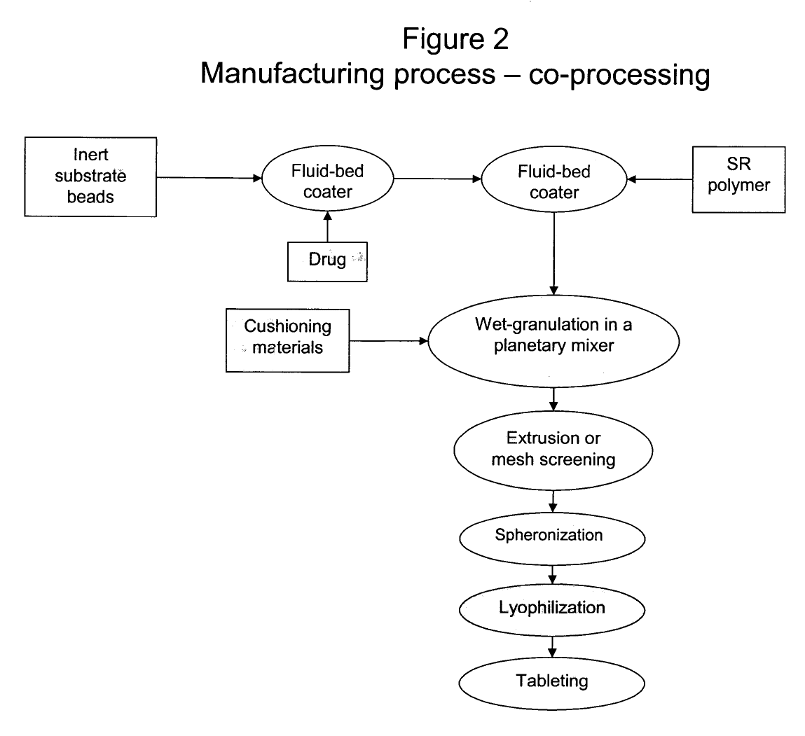

FIG. 3: Flow chart for the manufacturing of the non-co-processed Cushioning Beads™.

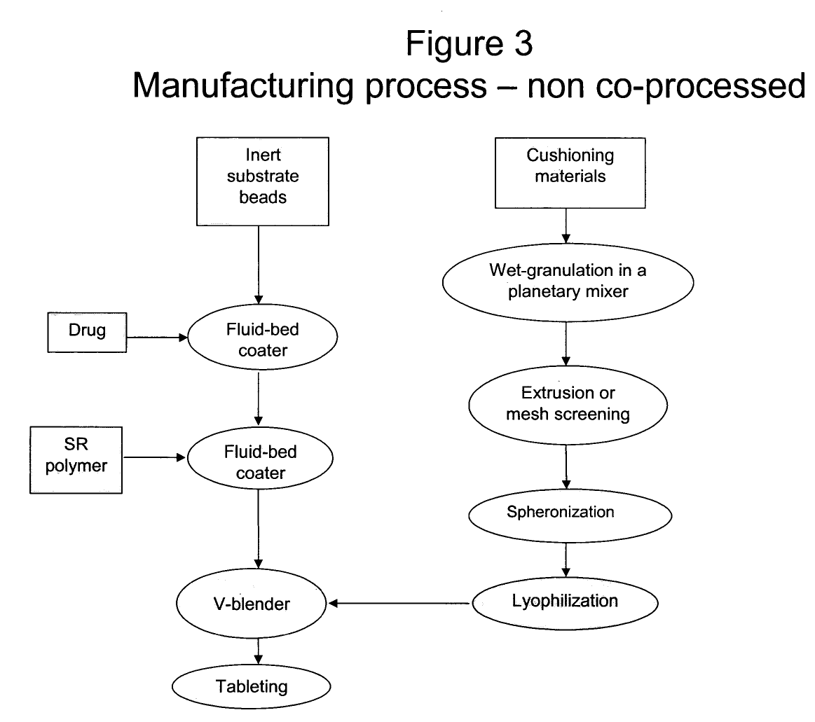

### EXAMPLES

Examples 1 and 2 are for Placebo Tablets to demonstrate the feasibility of the process and the necessary ranges for each step in the process.

Rapid Dispersion Placebo Tablets were prepared by the steps of:

i) Freeze-dried beads were milled and sieved through a 35 mesh US Standard screen;

ii) All excipients in Table I were accurately weighed out and passed through a screen;

iii) All excipients except the lubricant were mixed until uniform in a suitable container;

iv) The lubricant was added to the blend and the blend was mixed until uniform;

v) Tablets weighing about 350 mg were compressed on a Manesty D3B tablet press.

TABLE I

Tablet Formulation
(mg/tablet)

| | # 1 | # 2 |
|---|---|---|
| Mannitol | 250.3 | 222.3 |
| Xylitol | 63 | 56 |
| Freezed-dried Beads | 35 | 70 |
| Magnesium Stearate | 1.75 | 1.75 |

The disintegration time of tablets from example 2 was about 11-28 seconds, depending on how hard the tablets were compressed.

| | | |
|---|---|---|
| Mean | Mean | Mean In Vivo |
| Compression | Hardness | Dissolving |
| Force (kg) | (kp) | Time (sec.) |

Example 2

| | | |
|---|---|---|
| 52.5 | 0.6 | 11.7 |
| 72.3 | 1.3 | 16.0 |
| 110.5 | 1.7 | 28.0 |

Example 3

| | | |
|---|---|---|
| 61.1 | 0.6 | 11.7 |
| 75.2 | 1.1 | 14.3 |
| 113.4 | 1.9 | 23.7 |

Example 4

| | | |
|---|---|---|
| 60.1 | 0.6 | 9.3 |
| 76.8 | 1.0 | 15.7 |
| 122.8 | 1.8 | 25.5 |

Example 5

| | | |
|---|---|---|
| 56.4 | 0.6 | 8.7 |
| 77.6 | 1.0 | 17.0 |

Example 6

| | | |
|---|---|---|
| 52.9 | 0.5 | 9.7 |
| 83.8 | 1.0 | 10.0 |
| 126.3 | 2.0 | 16.0 |

Examples 3 thru 6 include active pharmaceutical ingredient. Examples 3, 4 and 5 contain Dextromethorphan and the active was taste masked.

Example 6 contains ascorbic acid and was not taste masked.

Rapid Dispersion Tablets containing active ingredient were prepared by the steps of:

vi) Freeze-dried beads were milled and sieved through a US Standard screen (refer to each formulation in Table II and III for screen size);

vii) All excipients in Table I were accurately weighed out and passed through a screen;

viii) All excipients except the lubricant were mixed until uniform in a suitable container;

ix) The lubricant was added to the blend and the blend was mixed until uniform;

x) Tablets weighing about 350 mg were compressed on a ManestyD3B tablet press.

TABLE II

| | Tablet Formulation | | |
|---|---|---|---|
| | (mg/tablet) | | |
| | # 3 | # 4 | # 5 |
| Dextromethorphan | 72.5 | 72.5 | 36.2 |
| Hydrobromide | | | |
| (taste-masked) | | | |
| Mannitol | 190.9 | 190.9 | 212.1 |
| Xylitol | 49 | 49 | 54.25 |
| Freezed-dried Beads | 35 | 35 | 43.75 |
| (screen size) | (#35) | (#60) | (#40) |
| Flavoring agent | — | — | 0.875 |
| Sweetener | — | — | 0.175 |
| Sodium Stearyl Fumarate | 2.6 | 2.6 | 2.625 |

TABLE III

| | Tablet |
|---|---|
| | Formulation |
| | (mg/tablet) |
| | # 6 |
| Ascorbic Acid | 46.34 |
| Mannitol | 191.33 |
| Xylitol | 50 |
| Freezed-dried Beads | 57 |
| (screen size) | (#35) |
| Flavoring agent | 2.5 |
| Sweetener | 0.33 |
| Sodium Stearyl Fumarate | 2.5 |

The disintegration time of tablets from example 3-6 was about 8-25 seconds, depending on how hard the tablets were compressed.

Materials and Methods

The method for production of the quick-dissolve tablet can follow four different routes and will ultimately yield the same desired outcome a tablet that disintegrates quickly in the oral cavity without the addition of liquid other than saliva and is capable of production on conventional manufacturing and packaging equipment.

The first proposed method is the direct blending of Cushioning Beads™ and active followed by compression using an appropriate amount of force to produce a tablet.

The second proposed method is to mix and wet granulate with a suitable binder the Cushioning Beads™ and active followed by compression using an appropriate amount of force to produce a tablet.

The third proposed method is to co-process the Cushioning Beads™ and active into a single bead that is then mixed, granulated, extruded, spheronized and freeze-dried as provided in detail in U.S. patent application Ser. No. 60/437,507.

Finally, the fourth proposed method follows the same process as described in U.S. patent application Ser. No. 60/437,507; however, the extrudate is freeze-dried and sized prior to tableting.

The active pharmaceutical compounds is not limited and can also include dietary supplements such as calcium, vitamins or minerals. In addition, the active ingredient can be a pro-drug.

The taste-masking can be accomplished by a number of methods including but not limited to coating, microencapsulation, coadmixture with flavors, sweetners or excipients that produce a cooling sensation in the mouth. Examples include sucrose, dextrose, sorbitol, mannitol, xylitol or any other pharmaceutically acceptable sugar, sugar alcohol or non-nutritive sweetner, alone or in combination.

Additional components can be added to improve functionality or patient compliance without effecting the fast-dissolve characteristic and include pigments, lakes or dyes or a suitable lubricant and glidant for appropriate manufacturability. These may include but are not limited to stearic acid, metallic stearates, such as magnesium or calcium stearate, hygrogenated fats, sodium stearyl fumarate, glycerly behenate and others.

Finally, the invention allows for the inclusion of appropriate adjunctive amounts of any suitable grade disintegrants of the classes of modified cellulose such as croscarmellose sodium, NF, modified starch such as sodium starch glycolate, NF or cross-link polyvunylpyrrolidone such as crospovidone, NF.

1. A method for the manufacture of a fast dissolve tablet that includes:
a) blending of highly-compactable filler in combination with a highly water-absorbing material and;
b) adding purified water to the mixture of highly compactable filler and highly water-absorbing material until granules are formed by visual inspection thus creating the cushioning component; and
c) the cushioning component is milled to a particle size of between 10-325 mesh (2000-45 micron); and
d) adding active-loaded beads to the milled cushioning component to create a mixture followed by an optional step of extrusion and spheronization and the option of including inactive ingredients to improve patient compliance, functionality or manufacturability; and
e) freeze-drying of the mixture of active-loaded beads and milled cushioning component with or without said extrusion and spheronization to create the Cushioning Beads™; and
f) compressing the Cushioning Beads™ into a fast dissolve tablet for treatment of a patient in need of said treatment.

2. A method for the manufacture of a fast dissolve tablet that includes:
a) blending of Avicel® PH101 and Ac-Di-Sol® and;
b) adding purified water to the mixture of Avicel® PH101 and Ac-Di-Sol® until granules are formed by visual inspection thus creating the cushioning component and;
c) the cushioning component is milled to a particle size of between 10-325 mesh (2000-45 micron); and
d) adding of active-loaded beads to the milled cushioning component to create a mixture followed by an optional step of extrusion and spheronization and the option of including inactive ingredients to improve patient compliance, functionality or manufacturability; and
e) freeze-drying of the mixture of active-loaded beads and milled cushioning component with or without said extrusion and spheronization to create the Cushioning Beads™; and
f) compressing the Cushioning Beads™ into a fast dissolve tablet for treatment of a patient in need of said treatment.

3. A method for the manufacture of a fast dissolve tablet that includes:
a) blending of Avicel® PH101 and Ac-Di-Sol® in a ratio that the mixture will have Ac-Di-Sol ranging from 5 to 90% by weight and;
b) adding of purified water to the mixture of Avicel® PH101 and Ac-Di-Sol® until granules are formed by visual inspection thus creating the cushioning component and;
c) the cushioning component is milled to a particle size of between 10-325 mesh (2000-45 micron); and
d) adding of active-loaded beads to the milled cushioning component to create a mixture followed by an optional step of extrusion and spheronization and the option of including inactive ingredients to improve patient compliance, functionality or manufacturability; and
e) freeze-drying of the mixture of active-loaded beads and the milled cushioning component with or without said extrusion and spheronization to create the Cushioning Beads™; and
f) compressing the Cushioning Beads™ into a fast dissolve tablet for treatment of a patient in need of said treatment.

4. A method for the manufacture of a fast dissolve tablet that includes:
a) blending of a highly-compactable filler in combination with a highly water-absorbing material and;
b) adding of purified water to the mixture of highly-compactable filler and highly water-absorbing material until granules are formed by visual inspection thus creating the cushioning component and;
c) the cushioning component is milled to a particle size of between 10-325 mesh (2000-45 micron); and
d) adding of active-loaded beads to the milled cushioning component to create a mixture followed by an optional step of extrusion and spheronization and the option of including nonactive ingredients to improve patient compliance, functionality or manufacturability; and
e) freeze-drying of the mixture of active-loaded beads and the milled cushioning component with or without said extrusion and spheronization until a LOD of 2-15% is achieved to create the Cushioning Beads™ followed by an optional step of extrusion and spheronization of the Cushioning Beads™; and
f) compressing the Cushioning Beads™ into a fast dissolve tablet for treatment of a patient in need of said treatment.

5. A method for the manufacture of a fast dissolve tablet that includes:
a) blending of Avicel® PH 101 and Ac-Di-Sol® and;
b) adding purified water to the mixture of Avicel® PH101 and Ac-Di-Sol® until granules are formed by visual inspection thus creating the cushioning component and;
c) the cushioning component is milled to a particle size of between 10-325 mesh (2000-45 micron); and
d) adding of active-loaded beads to the milled cushioning component to create a mixture followed by an optional step of extrusion and spheronization and the option of including inactive ingredients to improve patient compliance, functionality or manufacturability; and
e) freeze-drying of the mixture of active-loaded beads and milled cushioning component with or without said extrusion and spheronization until a LOD of 2-15% is achieved to create the Cushioning Beads™; and
f) compressing the Cushioning Beads™ into a fast tablet for treatment of a patient in need of said treatment.

6. A method for the manufacture of a fast dissolve tablet that includes:
a) blending of Avicel® PH101 and Ac-Di-Sol® in a ratio that the mixture will have Ac-Di-Sol ranging from 5 to 90% by weight;
b) adding of purified water to the mixture of Avicel® PH101 and Ac-Di-Sol® until granules are formed by visual inspection thus creating the cushioning component and;
c) the cushioning component is milled to a particle size of between 10-325 mesh (2000-45 micron); and
d) adding of active-loaded beads to the milled cushioning component to create a mixture followed by an optional step of extrusion and spheronization and the option of including inactive ingredients to improve patient compliance, functionality or manufacturability; and
e) freeze-drying of the mixture of active-loaded beads and milled cushioning component with or without said extrusion and spheronization until a LOD of 2-15% is achieved to create the Cushioning Beads™; and
f) compressing the Cushioning Beads™ into a fast dissolve tablet for treatment of a patient in need of said treatment.

7. A method for the manufacture of a fast dissolve tablet that includes:
a) blending of a highly-compactable filler in combination with a highly water-absorbing material and;
b) adding purified water to the mixture of highly-compactable filler and highly water-absorbing material until granules are formed by visual inspection thus creating the cushioning component and;
c) the cushioning component is milled to a particle size of between 10-325 mesh (2000-45 micron); and
d) adding more than one type of active-loaded beads to the milled cushioning component to create a mixture followed by an optional step of extrusion and spheronization and the option of including inactive ingredients to improve patient compliance, functionality or manufacturability; and
e) freeze-drying of the mixture of active-loaded beads and milled cushioning component with or without said extrusion and spheronization to create the Cushioning Beads™; and
f) compressing the Cushioning Beads™ into a fast dissolve tablet for treatment of a patient in need of said treatment.

8. A method for the manufacture of a fast dissolve tablet that includes:
a) blending of Avicel® PH101 and Ac-Di-Sol® and;
b) adding purified water to the mixture of Avicel® PH101 and Ac-Di-Sol® until granules are formed by visual inspection thus creating the cushioning component and;
c) the cushioning component is milled to a particle size of between 10-325 mesh (2000-45 micron); and
d) adding of more than one type of active-loaded beads to the milled cushioning component to create a mixture followed by an optional step of extrusion and spheronization and the option of including inactive ingredients to improve patient compliance, functionality or manufacturability; and
e) freeze-drying of the mixture of active-loaded beads and milled cushioning component with or without said extrusion and spheronization to create the Cushioning Beads™; and
f) compressing the Cushioning Beads™ into a fast dissolve tablet for treatment of a patient in need of said treatment.

9. A method for the manufacture of a fast dissolve tablet that includes:
a) blending of Avicel® PH101 and Ac-Di-Sol® in a ratio that the mixture will have Ac-Di-Sol ranging from 5 to 90% by weight and;
b) adding of purified water to the mixture of Avicel® PH101 and Ac-Di-Sol® until granules are formed by visual inspection thus creating the cushioning component and;
c) the cushioning component is milled to a particle size of between 10-325 mesh (2000-45 micron); and
d) adding of more than one type of active-loaded beads to the milled cushioning component to create a mixture followed by an optional step of extrusion and spheronization and the option of including inactive ingredients to improve patient compliance, functionality or manufacturability; and
e) freeze-drying of the mixture of active-loaded beads and the milled cushioning component with or without said extrusion and spheronization to create the Cushioning Beads™; and
f) compressing the Cushioning Beads™ into a fast dissolve tablet for treatment of a patient in need of said treatment.

10. A method for the manufacture of a fast dissolve tablet that includes:
a) blending of a highly-compactable filler in combination with a highly water-absorbing material and;
b) adding of purified water to the mixture of highly-compactable filler and highly water-absorbing material until granules are formed by visual inspection thus creating the cushioning component and;
c) the cushioning component is milled to a particle size of between 10-325 mesh (2000-45 micron); and
d) adding of more than one type of active-loaded beads to the milled cushioning component to create a mixture followed by an optional step of extrusion and spheronization and the option of including inactive ingredients to improve patient compliance, functionality or manufacturability; and
e) freeze-drying of the mixture of active-loaded beads and the milled cushioning component with or without said extrusion and spheronization until a LOD of 2-15% is achieved to create the Cushioning Beads™; and
f) compressing the Cushioning Beads™ into a fast dissolve tablet for treatment of a patient in need of said treatment.

11. A method for the of manufacture of a fast dissolve tablet that includes:
a) blending of Avicel® PH101 and Ac-Di-Sol® and;
b) adding purified water to the mixture of Avicel® PH101 and Ac-Di-Sol® until granules are formed by visual inspection thus creating the cushioning component and;
c) the cushioning component is milled to a particle size of between 10-325 mesh (2000-45 micron); and
d) adding of more than one type of active-loaded beads to the milled cushioning component to create a mixture followed by an optional step of extrusion and spheronization and the option of including inactive ingredients to improve patient compliance, functionality or manufacturability; and
e) freeze-drying of the mixture of active-loaded beads and milled cushioning component with or without said extrusion and spheronization until a LOD of 2-15% is achieved to create the Cushioning Beads™; and
f) and compressing the Cushioning Beads™ into a fast dissolve tablet for treatment of a patient in need of said treatment.

12. A method for the manufacture of a fast dissolve tablet that includes:
a) blending of Avicel® PH101 and Ac-Di-Sol® in a ratio that the mixture will have Ac-Di-Sol ranging from 5 to 90% by weight and;
b) adding of purified water to the mixture of Avicel® PH101 and Ac-Di-Sol® until granules are formed by visual inspection thus creating the cushioning component and;
c) the cushioning component is milled to a particle size of between 10-325 mesh (2000-45 micron); and
d) adding of more than one type of active-loaded beads to the milled cushioning component to create a mixture followed by an optional step of extrusion and spheronization and the option of including inactive ingredients to improve patient compliance, functionality or manufacturability; and
e) freeze-drying of the mixture of active-loaded beads and milled cushioning component with or without said extrusion and spheronization until a LOD of 2-15% is achieved to create the Cushioning Beads™; and
f) compressing the Cushioning Beads™ into a fast dissolve tablet for treatment of a patient in need of said treatment.
